(19) United States
(12) Patent Application Publication
(10) Pub. No.: US 2010/0228566 A1
(43) Pub. Date: Sep. 9, 2010

(54) Portable Vital Statistics Monitoring and Medication Dispensing System

(76) Inventors: Gloria Taylor, Miami, FL (US); Emerson Naylor, Tallahassee, FL (US); Deliena Stone, Tallahassee, FL (US); Evan Anderson, Tallahassee, FL (US)

Correspondence Address: THE PATENT GUILD, 313 COURTNEY SPRINGS CIRCLE, WINTER SPRINGS, FL, 32708, US

(21) Appl. No.: 12/400,255

(22) Filed: Mar. 9, 2009

(51) Int. Cl. G06Q 50/00; A61B 5/00; G06Q 10/00; G08B 23/00; G05B 19/00; G08B 21/00

(52) U.S. Cl. 705/3; 600/301; 700/215; 700/241; 700/242; 700/235; 340/573.1; 340/5.53; 340/540

(57) ABSTRACT

A portable vital statistics monitoring and medication dispensing system that provides personal healthcare management and daily prescription routines. The medication dispenser and vital statistics monitor combination incorporates a medication dispenser, a blood testing system, and other vital sign monitoring into a portable wireless device with customizable features. The medication dispenser and vital statistics monitor combination has the capability of notifying the user of the correct times to take medication and automatically dispensing the accurate prescription dosage. The medication dispenser and vital statistics monitor combination communicates the patient information using an onboard display, transmits the information to a local or distributed network, or prints/embosses/etches the information onto patient information cards, wristbands, or other information sheets.

### BACKGROUND OF THE INVENTION

1. Field of the Invention

The present invention relates to a system for dispensing pills, monitoring patient vital statistics and medication regimes, and providing patient status information.

It is to be understood that the term “user” may refer to the patient, that is the individual receiving the medication, or can be, for example, a caregiver, a pharmacist, physician, physician assistant, or patient relative. The term vital sign(s) refers to vital statistics.

Generally known medication-dispensing systems include the following U.S. Pat. No. 4,748,600 (Interactive drug dispenser); U.S. Pat. No. 5,200,891 (Electronic medication dispensing method); U.S. Pat. No. 5,408,443 (Programmable medication dispensing system); U.S. Pat. No. 5,611,912 (Medication compliance, coordination and dispensing system); U.S. Pat. No. 5,802,014 (Portable tablet reminder and dispensing system; U.S. Pat. No. 6,301,196 (Multiple alarm timepiece with pill compartments); U.S. Pat. No. 6,318,622 (Medication dispensing station); U.S. Pat. No. 6,401,991 (Computer timed locked medication container with individual compartments); U.S. Pat. No. 6,574,165 (Medication compliance apparatus); U.S. Pat. No. 6,594,549 (Web-enabled medication dispenser); U.S. Pat. No. 6,662,081 (Medication regimen container and system); U.S. Pat. No. 7,048,181 (Personal medication dispenser); U.S. Pat. No. 7,158,011 (Medication compliance device); U.S. Pat. No. 7,234,047 (System for dispensing pill or capsule-form medication in desired doses) and Patent Application Publications: US 2004/0179422 A1 (Pill dispensing reminder capable of communicating with a remotely situated computer); and US 2007/0036036 A1 (Pill timer).

Generally known patient drug regime compliance monitoring systems include the following U.S. Pat. No. 5,950,632 (Medical communication apparatus, system, and method; U.S. Pat. No. 5,967,975 (Home health parameter monitoring system); U.S. Pat. No. 6,824,152 (Communications system for an implantable device and a drug dispenser, and Patent Application Publications: US 2003/0086338 A1 (Wireless web based drug compliance system).

Generally known drug labeling and information systems include U.S. Pat. No. 4,918,604 (Prescription drug depiction and labeling system); U.S. Pat. No. 6,318,622 (Medication dispensing station).
Generally known printing/scanning/identification technology includes U.S. Pat. No. 3,898,922 (Printer for embossed card); U.S. Pat. No. 4,318,554 (Combined medical and/or informational identification credit card); U.S. Pat. No. 4,783,917 (Wristband); U.S. Pat. No. 5,193,855 (Patient and healthcare provider identification system), U.S. Pat. No. 7,073,717 (Portable printer and data entry device connected thereto assembly.

Despite the availability of the above individual components and systems, a need still exists to provide patient's a secure, portable, stand-alone device having the ability to manage their medication regime, perform various medical tests, and generate a print out (such as a patient information card, wristband, or information sheet) of the patient's current medical status during times when there is limited or no access to infrastructure resources such as electricity, printers, and communication networks, such as during emergencies or disasters or in remote geographical locations, during military actions, social events, or while providing routine medical services within an economically disadvantaged community.

### SUMMARY OF THE INVENTION

The portable vital statistics monitoring and medication dispensing system and method (referred to herein as the “VMonitor”) provided herein as the instant invention is an innovative electronic pill dispenser and vital statistics monitor combination that combines versatility with modern technology to provide simplicity for personal healthcare management and daily prescription routines. It incorporates a pill dispenser, a blood testing system, and other vital sign monitoring into a portable wireless device with customizable features. The VMonitor has the capability of notifying the user of the correct times to take medication and automatically dispensing the accurate prescription dosage. The VMonitor can be used to test the following patient vital signs: blood sugar, blood density, blood pressure, heart rate, O2 saturation, temperature. The VMonitor can also be configured, reconfigured, or customized to test additional patient vital signs as needed, such as to measure body weight, to communicate the patient information using an onboard display, or to transmit the information to a local or distributed network.

For security and safety precautions, the VMonitor may use biometric verification techniques (such as fingerprint or iris scanning), voice recognition, password protection, as well as token and/or smart card reading. The VMonitor uses a color LCD screen to display prescription and vital statistic information. In addition to its visual display, the VMonitor includes audio capabilities. The VMonitor also contains a scanner (such as a barcode scanner) to retrieve prescription information and may include an internal printer that prints out the vital statistic information, patient care information, pill dispensing information, and pill labeling information, as well as creates an informational card, wristband, or sheet. Additionally the VMonitor can provide users, physicians, caregivers, and family with periodic information updates and medical alerts.

### BRIEF DESCRIPTION OF THE DRAWINGS

The invention is explained below in view of the drawings:

FIG. 1 illustrates the VMonitor,

FIG. 2 illustrates a rear view of the VMonitor,

FIG. 3 is a block diagram of the VMonitor,

FIG. 4 is a block diagram of the VMonitor control unit,

FIG. 5 is a block diagram of the VMonitor communications module,

FIG. 6 is a block diagram of the VMonitor print module,

FIG. 7 is a block diagram of general usage procedures of the VMonitor,

FIG. 8 displays VMonitor general menu options,

FIG. 9 displays a patient wristband,

FIG. 10 displays a medication prescription label,

FIG. 11 displays a medication or vital statistics or patient information card/sheet.

### DETAILED DESCRIPTION OF THE INVENTION

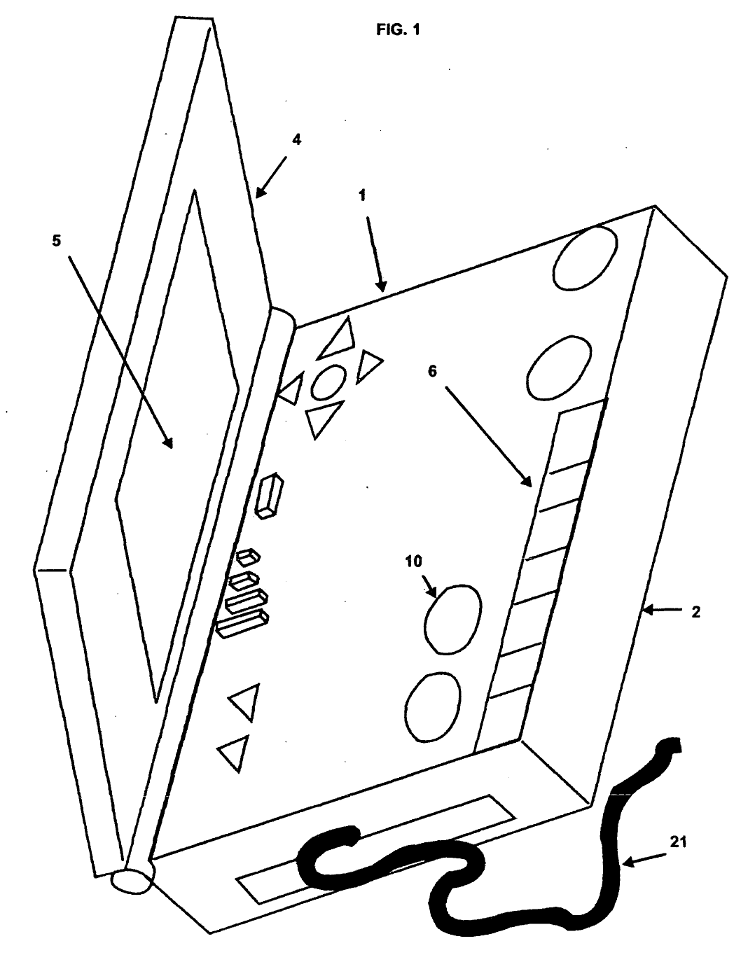

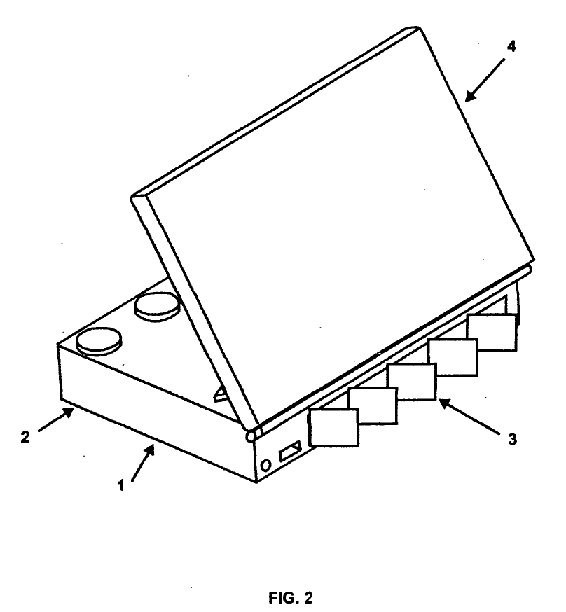

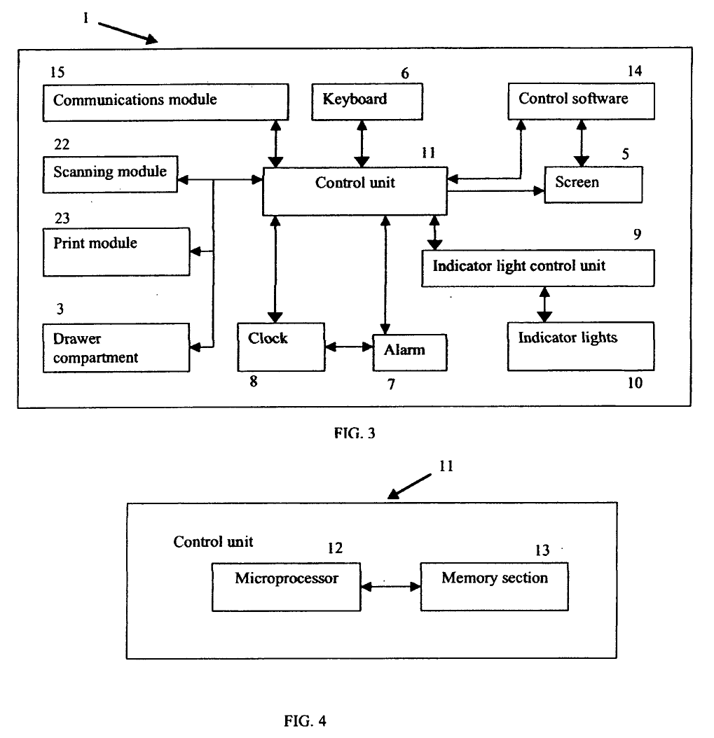

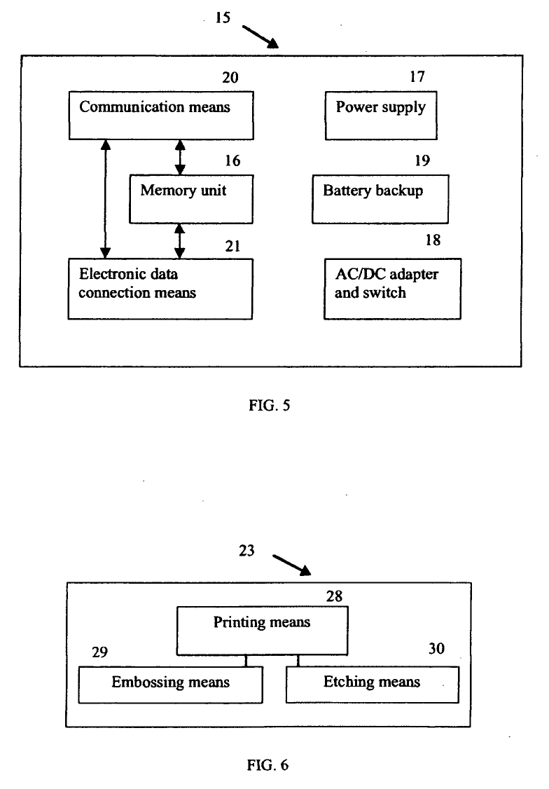

The preferred embodiment of the portable vital statistics monitoring and medication dispensing system and method (VMonitor) 1, as shown in FIGS. 1-11, includes a housing 2 having a plurality drawer compartments 3 arranged to contain and provide access to drugs, medication, and/or pills, a hinged cover 4 containing a color LCD screen 5 which is adapted to be folded down on the top of the housing 2. A keyboard 6 having a plurality of input/output buttons and switches is provided on the housing 2 to enter information necessary for the operation of the VMonitor 1. The interior of the housing 2 is arranged to contain and provide access to drugs, medication, and/or pills stored in the drawer compartments 3, the drugs, medication, and/or pills are taken on a specific time schedule or as needed. The drawer compartments 3, as shown in FIG. 2, may be positioned at the rear of the housing. Alternatively the drawer compartments 3 may be positioned on the sides or front of the housing 2. The VMonitor 1 includes an alarm 7, a clock 8, an indicator light control unit 9, and indicator lights 10. External devices such as a weight scale can be attached via wires or wireless connections.

The VMonitor 1 includes a control unit 11 containing a microprocessor 12 and a memory section 13. The control unit 11 controls the VMonitor 1 components in response to information from various units and modules, as well as instructions entered by operation of the keyboard 6 as described herein. The VMonitor 1 is controlled by control software 14 that interoperates with the control unit 11.

The VMonitor 1 further includes a communications module 15 which may, for example, communicate with other VMonitor units, a telephone interface modem, an external printer, an infrared control unit, and/or wireless devices (such as a Bluetooth enabled device). The communications module 15 also includes a memory unit 16, and a power supply system 17 that includes a standard AC/DC adaptor and associated switching 18. Preferably, the power supply system also includes a battery back-up 19 for example, a battery and charging unit to permit operation during power failures, while traveling or at remote locations. The VMonitor 1 is web-enabled via electronic communication means 20 for connecting to the Internet. The communication means 20 may be in the form of a wire communication device, such as a modem and its associated connection jack, or it may be in the form of a wireless communication device such as a Bluetooth device, a radio frequency device, or an infrared device.

The communications module 15 includes electronic data connection means 21 such as a wire, parallel ports, serial ports or USB ports for linking to a vital statistics monitoring and/or testing devices such as devices which test or sample or monitors the patient's blood sugar, blood density, blood pressure, heart rate, O2 saturation, temperature, and body weight. The preceding list is not exhaustive and it is envisioned additional vital statistics testing devices, beyond those listed, may be used in conjunction with the VMonitor 1.

The communication module's electronic data connection means 21 can also connect the VMonitor 1 to other devices such as other VMonitors, a computer, personal digital assistant, cell phone, WEB TV, a monitor, or other devices, and combinations thereof. For security and safety the VMonitor 1 may further include conventional biometric security components such as a fingerprint or iris scanner, voice recognition software, password protection software, and smart card or token readers. The VMonitor 1 may include other conventional computer hardware, including for example, extended memory chip or data storage module (e.g. RAM), a sound card, a video card, a printer cable, and an internal audio speaker.

An embodiment of the VMonitor 1 also may include a scanning module 22 including a scanner for reading and inputting data. The control unit 11 and control software 14 interoperate to control the scanning module 22. The scanning module 22 may be fixedly or removably connected to the housing 2 or to other modules in the VMonitor 1. For example, the scanning module 22 can be provided within the housing 2 or may be attached to the housing 2 via the electronic data connection means 21. The scanning module 22 may be utilized to scan data from information labels such as from patient information cards 24, wristbands 26, information sheets, or text documents, and to provide fingerprint or iris scanning. Additionally, the scanner module 22 may be used to scan pills or other objects (such as medical devices) to provide an image of the scanned object.

The VMonitor 1 further includes a print module 23 provided within the housing 2, the print module 23 includes printing means 28, such as a printer, for print information on patient information cards 24, wristbands 26, or information sheets as explained herein and is controlled by the control unit 11. The printing means 28 may further include embossing means 29, such as an embossing device, and may include etching means 30, such as an etching device to provide embossed or etched impressions on the selected medium including the patient information cards 24, wristbands 26, or information sheets.

The VMonitor control software 14 includes a medication dispensing computer program 31 (hereinafter sometimes referred to as the “Meds Program 31 ”) and a vital statistics monitoring computer program 32 (hereinafter sometimes referred to as the “Vitals Program 32”). The Meds Program and the Vitals Program provide instructions to the control unit 11 and utilize the appropriate memory available on the VMonitor 1.

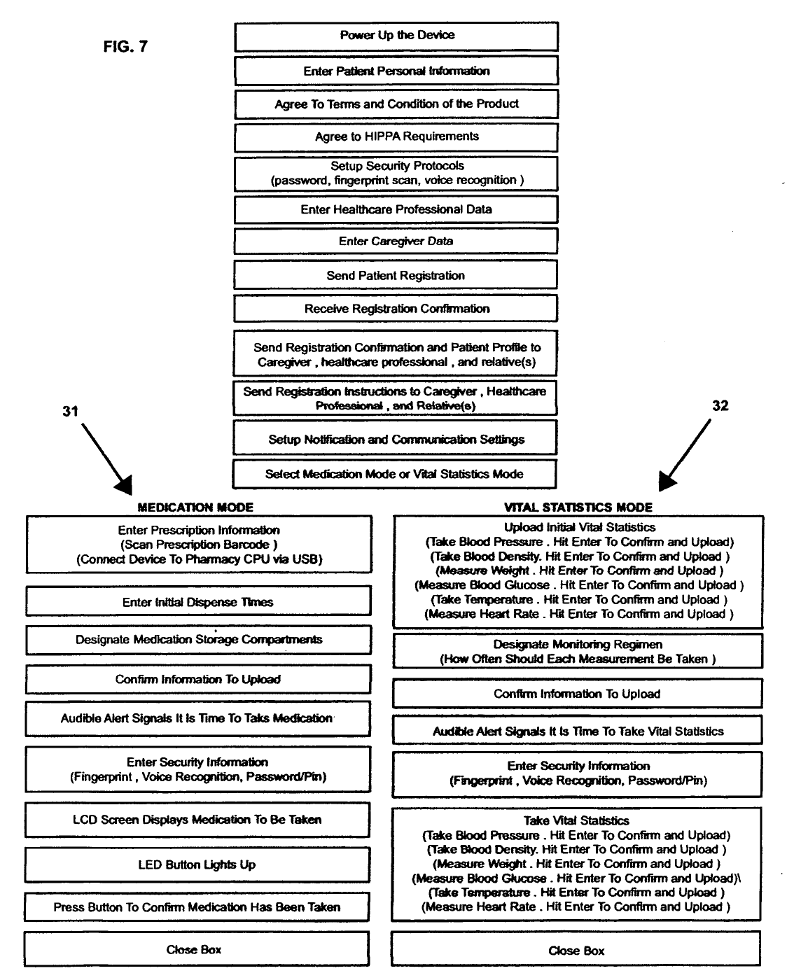

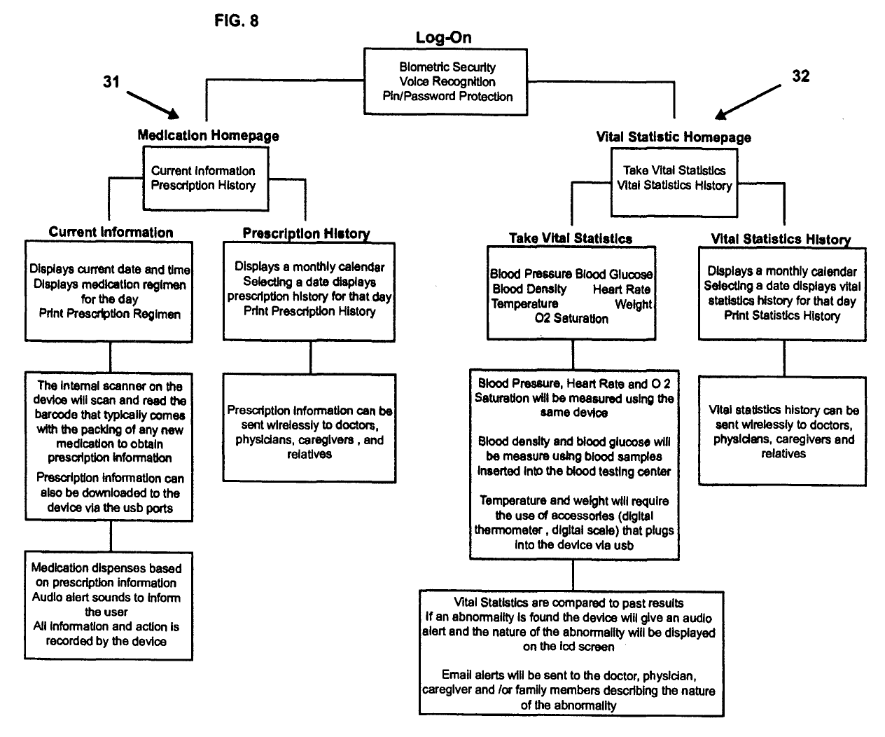

The Meds Program 31 can be either a pre-loaded program permanently stored in the control unit 11 or the user can load a downloaded or otherwise provided program into the control unit 11. The Meds Program 31 enables a variety of data relating to the patient and the patient's medication regimen to be entered. Initial data entry can be created prior to first use or upon connection to the Internet. Further data relating to the patient, the patient's medication regimen, and the medication may be updated as needed. As illustrated in FIG. 7 and FIG. 8, administrative data entry includes, for example, entering patient information (name, address and phone number), caregiver information, pharmacy information, physician information, insurance information, emergency contact information, medication names and descriptive information. The Meds Program 31 enables the user to input the desired medication regimen including the dispensing time and the compliance schedule.

Once the relevant Meds Program 31, patient information, and selected medication regimen has been entered into the Meds Program 31, the appropriate medication is dispensed in accordance with the medication regime schedule or other medication dispensation instructions. For example, patients may be provided medication based on a hourly, daily, weekly, or monthly schedule. Additionally the patient medication regime may be updated/modified based on information transmitted to the VMonitor 1 or the medication regime may be updated/modified based upon a recalculation of the patient's medication needs by the VMonitor 1. The VMonitor 1 can be programmed to automatically adjust the patient medication regime based the results of the vital statistics tests. For example, the dosage of drugs, medication, and/or pills may be increased or decreased based the results of the vital statistics tests or information transmitted to the VMonitor 1 by a physician, medical service provider, or caregiver.

The Vitals Program 32 can be either a pre-loaded program permanently stored in the control unit 11 or the user can load a downloaded or otherwise provided program into the control unit 11. The Vitals Program 32 manages the collection of a variety of data relating to the patient and the patient's physical condition including blood sugar, blood density, blood pressure, heart rate, O2 saturation, temperature, and patient body weight.

### Operation of the VMonitor

Use of the VMonitor 1 initiates with the user turning on the VMonitor 1 and performing the appropriate Log-On process. During initial usage, users may be directed via the LCD screen 5 to complete the VMonitor 1 setup, see FIG. 7, using the keyboard 6 and/or various buttons and switches. As indicated in FIG. 10, the VMonitor setup generally includes: entering patient personal information, agreeing to the terms and conditions of the product, agreeing to HIPPA requirements, setting up security protocols (such as passwords, fingerprint scans, and voice recognition setup), entering healthcare professional data, entering care giver data, sending appropriate patient registration information (to caregiver, healthcare professional, service providers, relatives, etc), receiving registration confirmation, sending applicable registration confirmation and appropriate patient profile information (to caregiver, healthcare professional, service providers, relatives, etc), sending applicable registration instructions to (to caregiver, healthcare professional, service providers, relatives, etc), and setting up notification and communication settings.

The Log-On process may singularly or collectively include the user providing the correct password and pin, token, smart card, or the user being confirmed via biometric scanning/voice recognition. After successfully completing the Log-On process, the LCD display 5 offers the user the option to select either the Medication Mode 31 (which takes the user to the Medication Homepage and employs the Meds Program 31) or the user selects the Vital Statistics Mode 32 (which takes the user to the Vital Statistics Homepage and employs the Vitals Program 32) as shown in FIG. 7.

In selecting the Medication Mode the user is taken to the Medication Homepage 31 and presented the choice of “Current Information” or “Prescription History”.

If the user selects “Current Information” the VMonitor 1 displays information such as the current date/time, the medication regime for the day, and presents the user an option to print the prescription regime. As shown in FIG. 7 and FIG. 8, in the Medication Mode 31 the user can enter prescription information by scanning the prescription from a coded prescription/medication information label/sheet 27 or by connecting to a pharmacy via the communication means 20, or the electronic data connection means 21 (such as the USB port) and downloading the applicable prescription information.

The medication is then dispensed based on prescription information; an audio alert may sound to inform the user; and the VMonitor 1 records all information and actions.

If from the Medication Homepage 31 the user chooses the “Prescription History” option a monthly calendar is displayed and the user can select a date and the VMonitor will display the prescription history for that day and allow the user to print the specified prescription history. The prescription information can also be sent wirelessly to doctors, physicians, caregivers, and relatives.

If, after completing the Log-On process, the user chooses the “Vital Statistic Homepage” option the user then receives the option to either “Take Vital Statistics” or view the “Vital Statistics History”.

If the user chooses to view the “Vital Statistics History” then the VMonitor 1 displays a monthly calendar and the user can select a specific day and view the vital statistics history for that day. Additionally users are provided the option to “Print Statistics History”. The statistics history can also be sent wirelessly to doctors, physicians, caregivers, and relatives.

If the after choosing the “Vital Statistic Homepage”, the user chooses the “Take Vital Statistics” option, the VMonitor 1 gives users the option to test any of the following: Blood Pressure, Blood Glucose, Blood Density, Heart Rate, Temperature, Weight, and O2 Saturation. The Blood Pressure, Heart Rate, and O2 Saturation may be measured using the same testing device. The Blood density and Blood Glucose may be measured using blood samples inserted into a blood-testing center or device. Temperature, weight, and other vitals statistics testing may require the use of accessories (such as a digital thermometer, or a digital scale) that plugs into the VMonitor 1 via electronic data connection means 21. Once the desired vital statistics are taken they are compared to past results. If an abnormality is found the VMonitor 1 can provide an audible alert and the nature of the abnormality can be displayed on the LCD screen 5. Also email alerts may be sent to the doctor, physician, caregiver and/or family members describing the nature of the abnormality.

When any of the above printing operations are selected, the VMonitor 1 screen displays printing options that are controlled by the print module 23 and allow users to select the appropriate print output. Information may be printed as text, embossed impression, or as an etching on the applicable selected printing medium such as patient information cards 24, wristbands 26, or information sheets.

Using the print module 23 and the printing means 28, users can create portable patient information cards 24, wristbands 26, or information sheets as needed, such as while traveling, without the hassle or worry of locating and configuring a printer. The patient information cards 24 may be provided as bar-coded information cards 25 which are readable by scanning devices, such as a scanner connected to a VMonitor 1.

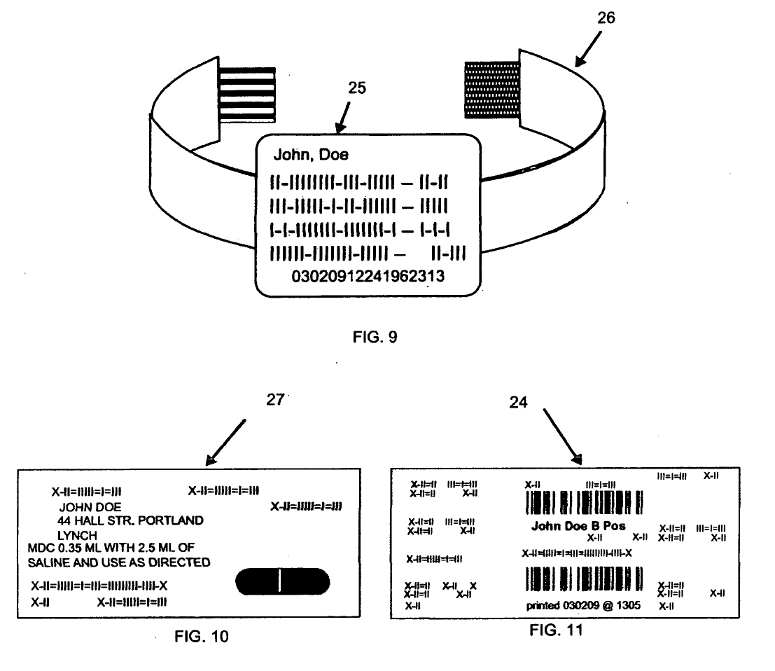

The patient information cards, wristbands, or information sheets as shown in FIG. 9 and FIG. 11, provides information such as patient name, address and phone number, caregiver information, pharmacy information, physician information, insurance information, emergency contact information, medication information such as a description of the medication and the patients medication regime.

A unique novelty of the VMonitor 1 is that after taking medication and/or performing vital statistic testing (even while traveling), patients can print out a patient information card, wristband, or information sheet that indicates the patient's most recent medical/medication history. Users (patients, caregivers, physicians, etc) can then carry the most recent patient information around with them as they perform their daily tasks. If a medical emergency arises, the medical support personnel, caregivers, physicians, etc can quickly determine the patient's most recent medical/medication history and render the most suitable assistance. For example, if, at a rest stop while traveling, a patient uses the VMonitor 1 to conduct tests of his/her blood pressure, heart rate, and O2 saturation after lunch and then prints out his/her most recent medical/medication history while still traveling, if the patient experiences medical difficulties, a quick review of the patient's information card, wristband, or information sheet with the most recent medical/medication history will apprise the medical support personnel, caregivers, physicians, etc of the patients most recent vital statistics. Since the VMonitor 1 includes a built-in printer, the medical support personnel, patient, caregiver, and/or physician does not need to wait until network connection or a printer can be located and configured to obtain the patient's medical information and most recent vital statistics. Even where patient data has been stored at a remotely accessible database it will not be as current as the information on the patient information card, wristband, or information sheet created from the vitals testing performed while the patient was at the rest stop.

Most significantly, since the patient can carry a patient information card, wristband, or information sheet that indicates the patient's most recent medical/medication history (such as data taken minutes earlier while at the rest stop), the patient does not need to carry around the VMonitor 1 or other similar device all through out the entire day. Additionally, patient information can be immediately transmitted to the appropriate personnel or medical support unit before the patient even arrives at the medical treatment location.

Additionally, the VMonitor 1 allows medical support personnel to test patients and create patient information cards, wristbands, or information sheets that indicate the patient's most recent medical/medication history (such as data taken minutes earlier) without the medical support personnel having to carry around a separate printer and its associated power supply, cords, etc. Exemplary medical support personnel users include the military services, the Veteran's Administration (VA), and civilian personnel.

During disasters and/or emergencies medical support personnel can use the VMonitor 1 to perform on-the-spot medical testing, prove the patient with the appropriate medication, and simultaneously provide the patient with a patient information card, wristband, or information sheet which has accurate and up-to-date medical/medication information. When the patient obtains supplemental or follow-up medical service, the patient can present the service provider the patient information card, wristband, or information sheet which will explain the emergency medical services the patient received as well as provide the record of patient's medical/medication information taken at the time of the emergency. The VMonitor can also be used as described above during military actions, social events, or while providing routine medical services within an economically disadvantaged community where there is limited access to support equipment such as printers, scanning devices, and medical testing equipment.

In geographical regions or locations where access to electricity and other technical infrastructure components is limited, the built-in components of the VMonitor 1, such as the printer and scanner, allows medical support personnel to perform on-the-spot medical testing, provide the patient with the appropriate medication, and simultaneously provide the patient with a patient information card, wristband, or information sheet which has accurate and up-to-date medical/medication information, despite the minimal or non-existent infrastructure. The VMonitor can provide education on various health issues via its screen or printouts or by electronically transmitting the information to a suitable electronic information receiving device. For example, VA patients can receive educational information at their convenience via the VMonitor.

For users who are visually impaired the print module 23 can create a printout that includes an embossed or etched impression. Additionally, the embossed or etched impression outputs of the VMonitor 1 can provide additional security of the patient information if an embossed/etched patient information card, wristband, or information sheet is created which can only be read by devices calibrated to appropriately comprehend the information encoded by the embossing such as another VMonitor 1. In these situations the second can scan the embossed/etched patient information card, wristband, or information sheet created by the first VMonitor 1 and import the appropriate data. The first and second. VMonitors can be calibrated to read distinguishing features of the embossed/etched patient information card, wristband, or information sheet and use this information as a security feature to protect the patient data and to control access to the VMonitor units.

Additionally, the VMonitor can be configured for use by children via the software which can provide a child-friendly user interface such as using icons which designate VMonitor functions and automatic or semi-automatic preset groups of functions. For example, the user interface can be programmed to visually display icons or images which show a child how to take body temperature (his/her own or that of another person) and transmit it to their parent and/or appropriate person or data receiving center. Using the VMonitor parents and/or the appropriate personnel can monitor and track the medical and medication history of the child or other user at a remote location such as an assisted living facility. If there are any irregularities the Vmonitor can be programmed to notify the appropriate personnel of the patient's status via an audible alarm, email/electronic messaging, or even by flashing the VMonitor indicator lights, such as with a Morse code sequence.

While various embodiments of the present invention have been shown and described herein, it will be obvious that such embodiments are provided by way of example only. Numerous variations, changes and substitutions may be made without departing from the invention herein. Accordingly, it is intended that the invention be limited only by the spirit and scope of the appended claims.

What is claimed is:

1. A vital statistics monitoring and medication dispensing system comprising:
a housing including drawer compartments for storing and dispensing medication;
a display screen attached to the housing;
a keyboard on the housing and having a plurality of input/output buttons and switches;
a control unit within the housing;
a data storage module within the housing;
an integrated print module within the housing;
a communications module within the housing;
at least one vital statistics monitoring device for testing blood sugar, blood pressure, heart rate, O2 saturation, temperature, and/or body weight; and
control software for providing control and interoperability between the housing, the display screen, the keyboard, the control unit, the data storage module, print module, the communications module, and the at least one vital statistics monitoring device.

2. The vital statistics monitoring and medication dispensing system of claim 1 further including a scanning module provided within the housing, the scanning module reading and inputting data as determined by the control software.

3. The vital statistics monitoring and medication dispensing system of claim 1 wherein the print module includes printing means that embosses or etches patient medication regime data, patient vital statistics data, and patient administrative data onto a patient information card, wrist band, or information sheet.

4. The vital statistics monitoring and medication dispensing system of claim 3 further including at least one security feature wherein users cannot log onto the system unless their identification is successfully confirmed from the patient administrative data embossed/etched on the patient information card, wrist band, or information sheet.

5. The vital statistics monitoring and medication dispensing system of claim 2 wherein the communications device can wirelessly send out vital statistics information, prescription information, medication regime history information, and email alerts of abnormalities in the vital statistics information.

6. The vital statistics monitoring and medication dispensing system of claim 5 wherein the medication regime data is inputted by scanning a medication information label, a patient information card, wristband, or information sheet containing patient medication regime data and/or patient vital statistics data.

7. The vital statistics monitoring and medication dispensing system of claim 1 wherein at a predetermined time the display screen displays an image or message indicating medication to be taken.

8. The vital statistics monitoring and medication dispensing system of claim 1 wherein at a predetermined time an audible alert signals medication is to be taken.

9. The vital statistics monitoring and medication dispensing system of claim 1 wherein vital statistics information is compared to past results and if an abnormality is found and audible alarm is given and the nature of the abnormality is presented on the display screen or transmitted to a caregiver, a pharmacist, physician, physician assistant, or patient relative.

10. The vital statistics monitoring and medication dispensing system of claim 2 further including at least one security feature selected from the group consisting of fingerprint scanning, iris scanning, and voice recognition,
wherein users cannot log onto the system unless their identification is successfully confirmed.

11. The vital statistics monitoring and medication dispensing system of claim 2 wherein the housing drawer compartments open and close independently according to a medication regime to allow dispensing of medication and refilling of the drawer compartments.

12. The vital statistics monitoring and medication dispensing system of claim 11 wherein the medication regime data is inputted by scanning an embossed/etched card containing patient medication regime data or patient vital statistics data.

13. The vital statistics monitoring and medication dispensing system of claim 12 wherein the embossed/etched card is generated by a vital statistics monitoring and medication dispensing system.

14. A method for providing patient vital statistics monitoring and medication dispensing comprising:
providing a housing including compartments for storing and dispensing medication;
a display screen attached to the housing;
providing a keyboard on the housing and having a plurality of input/output buttons and switches;
providing a control unit within the housing;
providing a data storage module within the housing;
providing an integrated print module within the housing;
providing a communications module within the housing;
and providing control software for providing control and interoperability between the housing, the display screen, the keyboard, the control unit, the data storage module, integrated print module, and the communications module,
inputting patient administrative data;
inputting patient medication regimen data;
performing tests of selected patient vital statistics;
generating a patient information card, wrist band, or information sheet using the integrated print module;
generating a medication dispensing schedule based on patient administrative data, patient medication regimen data, and tests of selected patient vital statistics; and
dispensing medication based on the medication dispensing schedule.

15. The method of claim 14 wherein the medication regime data is inputted by scanning a medication prescription label.

16. The method of claim 14 wherein the patient administrative data is inputted by scanning a patient information card, wristband, or information sheet.

17. The method of claim 14 wherein the medication regime data is inputted by scanning an embossed/etched card containing patient medication regime data or patient vital statistics data generated by a vital statistics monitoring and medication dispensing system.

18. The method of claim 14 further including transmitting current and/or historical medical data to a medical facility and/or medical treatment personnel prior to the patient's arrival to provide quicker triage and treatment.

19. The method of claim 14 further including providing indicator lights on the housing and flashing the indicator lights to indicate the status of the patient.

20. A vital statistics monitoring and medication dispensing system comprising:
a housing including drawer compartments for storing and dispensing medication, wherein the housing drawer compartments open and close independently according to medication regime data to allow dispensing of medication and refilling of the drawer compartments,
a display screen attached to the housing;
a keyboard on the housing and having a plurality of input/output buttons and switches;
a control unit within the housing;
a data storage module within the housing;
an integrated print module within the housing;
a communications module within the housing;
at least one vital statistics monitoring device attached to the housing, the device being useable for testing blood sugar, blood pressure, heart rate, O2 saturation, temperature, and/or body weight;
a scanning module provided within the housing, the scanning module reading and inputting data as determined by the control software wherein the medication regime data controlling the opening and closing of the drawer compartments is inputted by scanning a medication prescription label;.
control software for providing control and interoperability between the housing, the display screen, the keyboard, the control unit, the data storage module, print module, the communications module, and the at least one vital statistics monitoring device; and
at least one security feature selected from the group consisting of fingerprint scanning, iris scanning, and voice recognition wherein users cannot log onto the system unless their identification is successfully confirmed using the security feature,
wherein the communications device can wirelessly send vital statistics information, prescription information, medication regime history information, and email alerts of abnormalities in the vital statistics information,
wherein at a predetermined time the display screen displays an image or message indicating medication to be taken,
wherein vital statistics information is compared to past results and if an abnormality is found and audible alarm is given and the nature of the abnormality is presented on the display screen.
